(19) United States
(12) Patent Application Publication
(10) Pub. No.: US 2023/0137048 A1
(43) Pub. Date: May 4, 2023

(54) Devices and Methods for Reducing Transmission of Pathogens

(71) Applicant: William PETTY, Gainesville, FL (US)

(72) Inventor: William PETTY, Gainesville, FL (US)

(21) Appl. No.: 17/770,835

(22) PCT Filed: Jun. 29, 2021
(86) PCT No.: PCT/US2021/039636

§ 371 Date: Apr. 21, 2022

Related U.S. Application Data
(60) Provisional application No. 63/052,235, filed on Jul. 15, 2020.

(51) Int. Cl. A61B 5/11; A61B 5/00; G08B 21/24

(52) U.S. Cl. CPC A61B 5/1126; A61B 5/1122; A61B 5/486; A61B 5/681; A61B 5/6824; A61B 5/7405; A61B 5/742; A61B 5/7455; G08B 21/24; A61B 2562/0219

(57) ABSTRACT

This present invention relates to a monitoring device used to reduce touching of a wearer's head or neck. The monitoring device has an accelerometer that detects motion of the hand to the head and/or neck. The monitoring device can reduce the frequency of touching by issuing alerts to the wearer.

### CROSS-REFERENCE TO RELATED APPLICATION

This application claims priority to U.S. Provisional Patent Application No. 63/052,235, filed Jul. 15, 2020, which is incorporated herein by reference in its entirety.

### BACKGROUND OF THE INVENTION

Pathogens, including bacteria and viruses, can be transmitted by self-inoculation in which, for example, an individual's hands become contaminated with a pathogen and then the hands touch the individual's mouth, nose, and/or eyes. Pathogens can be spread after touching a contaminated surface and then touching one's face. It has been shown that on average an individual touches their head and face between 10 and 22 times an hour. The touching can involve one hand or both and can include, for example, rubbing, scratching, or grooming, or even merely be a subconscious habit. Regular handwashing and limiting hand contact with the face are critical factors in decreasing the propagation of infections.

Many respiratory viruses, including measles, adenovirus, coronavirus, rhinovirus and influenza, have high infectivity rates. The viruses are primarily transmitted by respiratory droplets, but viruses can be transmitted by direct contact such as with handshaking and they may remain infectious on inert surfaces, such as door handles and countertops, for many hours to days, precipitating the need to keep hands away from the face. Whether in a hospital, commercial office or residential facility, frequent face touching, particularly during periods of seasonal outbreak, presents a tremendous risk of acquisition and transmission of the pathogens.

Beginning in the 1960s, a variety of human-infecting coronaviruses have been identified. From the family Coronaviridae, these viruses primarily infect the upper respiratory and gastrointestinal tracts. Whilst many such infections are mild and routinely include the common cold, for example, far more pathogenic and potentially lethal strains exist, including SARS, MERS, and the 2019 outbreak strain of SARS-CoV-2, (2019-nCov or COVID-19).

SARS (Severe Acute Respiratory Syndrome), MERS (Middle East Respiratory Syndrome) and SARS-CoV-2 (SARS-related Coronavirus 2) are all highly pathogenic human coronaviruses responsible for acute and chronic diseases of the respiratory, hepatic, gastrointestinal and neurological systems. It is thought that each virus emerged from animal reservoirs and transferred to humans, thereby resulting in human epidemics, with the outbreak of SARS in 2002, MERS in 2012, and SARS-CoV-2 in late 2019.

Coronaviruses are enveloped single-stranded RNA viruses named so for their crown-like surface structure composed of spike (S), envelope (E), membrane (M) and nucleocapsid (N) proteins. The spike protein in particular is responsible for the action of entering a host cell, wherein the coronavirus is able to transcribe its RNA for intracytoplasmic replication. Indeed, coronaviruses have a unique ability to replicate and survive in the intracellular space of a macrophage, whereby multiple encoded interferon antagonists are thought to hinder the activation of type I interferon (IFN) and interferon stimulated genes (ISGs), dampening the host immune response and contributing to the resultant pathogenesis of the virus (Rose et al. 2010, Journal of Virology 84 (11): 5656-5669).

Upon genome replication and polyprotein formation, the viruses assemble and are released from the infected cell to further disseminate. Transmission between hosts is considered to occur primarily by contact with respiratory droplets infected with such viral particles, generated through sneezing and coughing (CDC.gov, 2020).

Coronaviruses can emerge from animal reservoirs to cause significant epidemics in humans, as exemplified by Severe Acute Respiratory Syndrome coronavirus (SARS-CoV) in 2002-2003 and Middle East Respiratory Syndrome coronavirus (MERS-CoV), which was recognized as an emerging virus in 2012, each of which resulted in over 8000 infections and 774 deaths, and 2500 infections and 862 deaths, per respective outbreak (WHO, 2020). Declared a global emergency by the World Health Organisation (WHO), the newly discovered and rapidly disseminating SARS-CoV-2, sharing ˜70% genetic similarity to the SARS-CoV, is likely to have similar epidemiological characteristics and thus presents a pressing area of healthcare concern. Crucially, there are no vaccines or antiviral drugs suitable for the prevention or treatment of human coronavirus infections at this time (Habibzadeh & Stoneman 2020, Int J Occup Environ Med 11 (2): 65-71).

A SARS-CoV-2 health emergency evidences a lack of effective virus-specific treatments or vaccines, which thus leads to a high unmet need for the protection of high-risk populations, including the elderly, health care workers and patients in acute danger of nosocomial transmission of SARS-CoV-2, or in other confined spaces, such as during quarantine settings.

Without a viable antiviral or vaccine currently approved, there exists a particular need for safe and effective methods of preventing pathogen transmission, particularly transmission that results in self-inoculation. Of particular need is a device that can teach or train an individual to reduce the number of times or even stop random or unconscious touching of the face and head.

Accordingly, a need exists for a device for detecting and notifying an individual of hand to face touching to limit self-inoculation and the propagation of a pathogen infection. Such notification can be effective in avoidance-behavior training

The compass application on a smartphone knows the direction a phone is pointing. A stargazing application on a smartphone knows where the camera lens is pointing in the sky to display the constellations in that area. Smartphones and other mobile technologies identify their orientation with an accelerometer, a small device that uses axis-based motion sensing devices. For example, Apple, Inc. has included an accelerometer in every generation of iPhone, iPad, and iPod touch, as well as in every iPod nano since the 4th generation. Along with orientation view adjustment, accelerometers in mobile devices can also be used as pedometers.

Devices having accelerometers incorporated therein and/or associated therewith have been described in, for example U.S. Pat. Nos. 7,306,567; 7,855,936; 8,265,900; 9,924,900; 10,401,800; 10,573,164 and 10,594,846.

According to Ryan Goodrich, of LiveScience in October, 2013 the quantified-self movement, a term coined by Gary Wolf and Kevin Kelley of Wired Magazine, refers to the increasing use of technology to collect data about oneself. These technologies, such as smartphone apps, GPS devices, and physical activity trackers with accelerometers allow individuals to track aspects of their daily lives, including their total activity, number of steps, food they eat, amount of sleep, heart rate, and mood. Such tracking not only allows individuals to learn more about themselves but can also help them take action to become healthier and improve their lives, according to the movement's followers.

The goal of the described invention is specifically to improve health of individuals by reducing the incidence of common infectious diseases such as the common cold and influenza.

### BRIEF SUMMARY OF THE INVENTION

The present invention is directed to monitoring devices to be worn on or near the hands of an individual, as well as the use of these devices. The monitoring devices can be worn on the forearm, wrist or hand. In certain embodiments, the monitoring devices can resemble, for example, a wrist band or a bracelet. Alternatively, the devices can resemble a ring or finger cuff worn on one or more fingers. A monitoring device can also be incorporated with another object, such as a wrist watch.

The monitoring device can include at least one accelerometer that can detect movement of the monitoring device, caused by movement of the hand or wrist of the individual. Thus, movement of the monitoring device can be interpreted as corresponding to movement of the hand. The monitoring device can also provide at least one of a visual, audible, and haptic signal indicative of a hand location detected by the accelerometer. If the accelerometer detects or senses movement that the microcontroller interprets is approaching or in proximity to the face or head, a feedback mechanism on the device emits the signal to notify and discourage the individual from continuing the motion of touching the head or face.

A single monitoring device can be worn. Alternatively, two monitoring devices can be worn, one on each forearm, hand or wrist. When either hand is moved towards the head or face, a signal can be emitted from one or the other monitoring device. There are, however, normal motions that necessitate moving both hands towards the face, but not for the purpose of touching the face. For example, lifting an object towards or above the head can bring the hands close to the face. In one embodiment, monitoring devices worn on or near each hand are in operable communication, such that the monitoring devices can communication with each other. In a further embodiment, the monitoring devices can be programmed to recognize one or more specific movements in which both hands are moved towards or in proximity to the face, so that such movement does not trigger the feedback mechanism and a signal being sent to the individual. If one hand is raised or makes a motion indicative of touching the head or face, the monitoring device can send a signal.

Preferred embodiments of the monitoring device have an accelerometer that detects or senses the magnitude and direction of hand motion. In one embodiment, one or more 2-axis or 3-axis accelerometer(s) is/are used to detect hand motion. The monitoring device can include a microcontroller operably connected to the one or more accelerometers. The microcontroller can be programmed to establish base-line boundaries or parameters for hand motion and orientation in the space or area around the individual. In a particular embodiment, the monitoring device is programmed with customized to distinct movement patterns of the individual that are directed towards the face region. For example, the monitoring device can be programmed to cause the feedback mechanism to send one or more signals or notifications if a hand is raised past a pre-programmed height or if one or both hands are moved outside of the pre-programmed parameters or boundaries. The device can also be programmed to distinguish between eating and other non-eating touches to the face such that eating does not cause a signal to be sent.

The feedback mechanism can send one or more signals to the individual that can be audible, visual, haptic, or some combination thereof. For example, the feedback mechanism can send a signal that an individual perceives as a buzz, vibration, sound, electric shock, light, or some combination thereof.

A wearable monitoring device can further include an interface operably connected to the microcontroller that permits the individual to control the type and intensity of the one or more signal or notification. The interface can also be used to turn off the signal or notification for a period of time, such as, for example, when eating or grooming.

### BRIEF DESCRIPTION OF THE FIGURES

FIGS. 1A and 1B illustrate embodiments of a monitoring device, according to the subject invention, configured to be worn on the wrist (FIG. 1A) or the finger (FIG. 1B).

FIGS. 2A and 2B illustrate a method of wearing two monitoring devices that are paired with each other. The paired monitoring devices transmit a position information signal to each other, as indicated by the dashed lines. FIG. 2A illustrates the paired monitoring devices being moved together to perform a motion that does not elicit a signal from the monitoring devices. FIG. 2B illustrates one of the paired monitoring devices moving towards the face, which causes a signal to be emitted by that monitoring device.

FIG. 3 shows a schematic of the components and operation of one embodiment of monitoring device, according to the subject invention.

FIG. 4 shows one embodiment of the general circuitry for a paired monitoring device of the subject invention, including an amplifier to amplify the information from the accelerometer, a transmitter for relaying a positional information signal and a receiver for receiving a positional information signal from another monitoring device.

### DETAILED DESCRIPTION OF THE INVENTION

The present invention is directed to a monitoring device to be worn on or near the hand of an individual, and its use to prevent, reduce and/or discourage hand to face touching that could transmit a pathogen. Thus, the device could be worn around the wrist similar to, for example, a wrist watch or bracelet or on one more fingers, similar to a ring or finger cuff.

As used herein, reference to on “or near” a hand or in the “proximity” of a hand means within 18 inches, such as within 12 inches, within 8 inches or within 6 inches, of the tip of the fingers on the hand.

In one embodiment, the monitoring device utilizes at least one accelerometer in order to precisely track the hand movements of the individual. The accelerometer can provide a signal indicative of a location or orientation of an individual hand in space. If movement is detected that indicates the hand is approaching, touching or in proximity to the face, the individual receives a signal or notification from the device.

In one embodiment, the invention provides a monitoring device, configured to be worn on or in proximity to a hand, comprising: at least one accelerometer that senses motion of the hand; a microcontroller, in operable communication with the accelerometer, programmable with one or more boundaries and that receives information from the accelerometer pertaining to the motion of the hand; a transmitter, in operable communication with the microcontroller, for transmitting a position information signal pertaining to the location of the monitoring device; and a feedback mechanism controlled by the microcontroller that provides a signal when the microcontroller determines that the hand approaches or passes a programmed boundary.

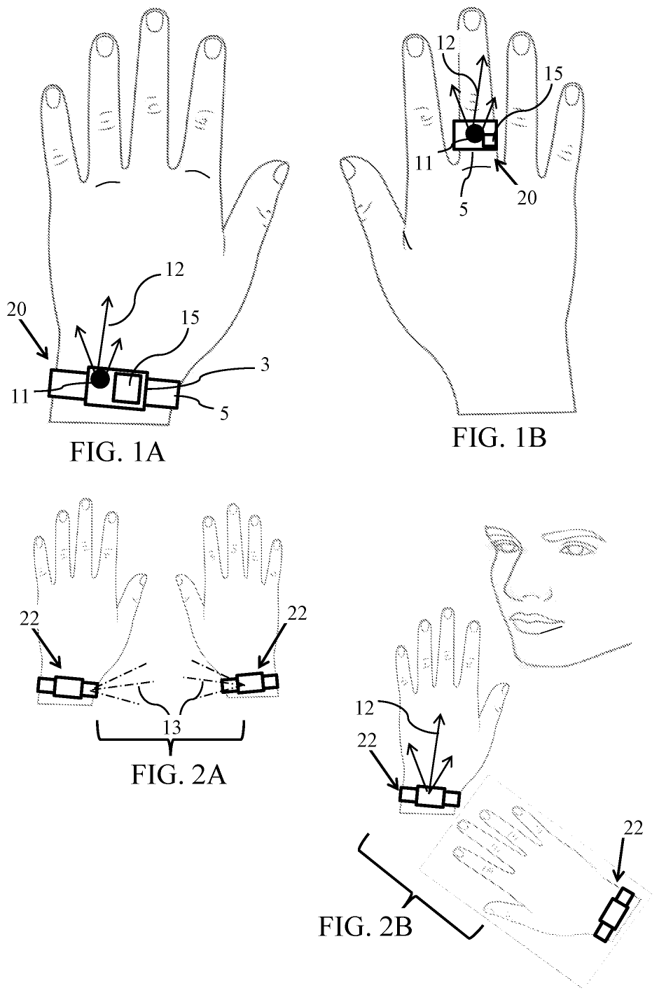

In one embodiment, a monitoring device 20 is worn on or near a hand. The monitoring device can have at least one, preferably at least two, accelerometers 7. The accelerometers can be in operable communication with a microcontroller 9 that receives information from the accelerometers corresponding to motion and then processes the information to determine the location of the monitoring device, which corresponds to the location of the hand in the space or area around an individual. The position of the hand can be further related to its position relative to the body of the individual. More specifically, the position of the hand can be determined relative to the head and face of the individual. The microcontroller can be programmed with pre-defined boundaries and when the hand moves beyond the boundaries, a signal 12 is provided to the individual. In one embodiment, the microcontroller is programmed to provide a signal when the hand moves near the face or head. FIGS. 1A and 1B illustrate an embodiment of a single monitoring device that can be worn on the wrist or on a finger.

In an alternative embodiment, an individual can wear two monitoring devices, a first monitoring device on or near one hand and a second monitoring device on or near the other hand. The first and second monitoring devices can be worn on the wrists or fingers. In one embodiment, the monitoring device can be worn on the wrist and the other on a finger. FIGS. 2A and 2B illustrate non-limiting examples of a first and second monitoring device worn on or near each hand. If either hand is moved towards the face or head a signal will be emitted to alert the wearer.

In a further embodiment, the first and second monitoring devices 20 are paired so that they communicate with each other. In one embodiment, each of the paired monitoring devices 22 comprise a transmitter 18 that can transmit or otherwise emit position information signals 13, continuously or at intervals. Position information signals can include, for example, radio, sound, light, infrared, and any other types of signal that can be sent and received by each device. In a further embodiment, each of the paired monitoring devices has a receiver 19 for receiving the position information signal from the other monitoring device paired therewith. The positional information signal transmitted by one monitoring device can provide location and/or proximity information to be received by the other monitoring device. In a further embodiment, the paired monitoring devices can be programmed to recognize when both hands are arranged in a pre-defined orientation and moving towards or in proximity to or above the head or face. In one embodiment, the microcontroller is programmed to compare the location information received from the one or more accelerometers and compare that information to the position information signal received from one other monitoring device. In comparing the location information and the position information signal the microcontroller can then determine if the hands are arranged in a pre-defined orientation. When performing those specific motions, requiring both hands in a pre-defined orientation, a signal will not be sent by the monitoring devices. In other words, paired monitoring devices can be programmed to disregard motion or actions that include both hands making specific pre-defined motions programmed into the microcontroller. By way of example, lifting something above the head or moving an object close to the face but not touching the face, when performed with both hands in a specific position, such as side-by-side, will not cause the paired monitoring devices to emit a signal.

FIG. 2A illustrates an example of paired monitoring devices emitting positions signals 13 to each other. As shown in FIG. 2A, when the hands are moved together towards the face or above the head they do not emit a signal alerting the individual. When the hands are moved back below the head and face the monitoring devices can be reset and will send a signal if one or the other hand moves alone or singly towards or in proximity to the head or face. FIG. 2B illustrates how a monitoring device can emit a signal if only one hand is moved towards the head or face.

In one embodiment, a monitoring device 20 comprises a housing 3 containing one or more accelerometers 7 that detect the motions of a hand and provide information to a microcontroller 9 programmed to determine the orientation and position of the hand in space and relative to the head and face of the individual. In other words, the monitoring device can determine the position of one or more hands relative to the space around the body of an individual, in particular the space around the head and face.

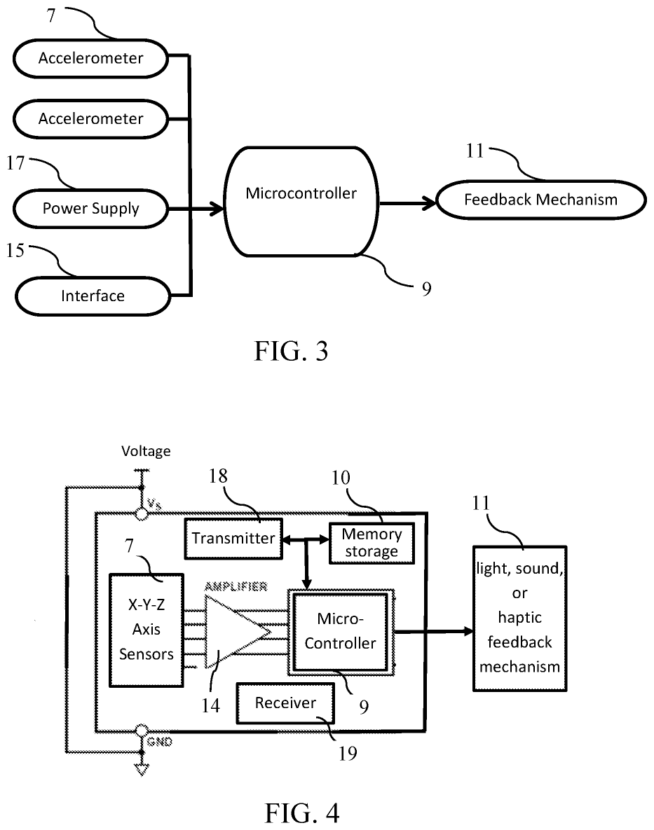

An accelerometer is a sensor that measures the acceleration forces acting on an object. It is known in the art that by measuring forces acting on the object in more than one direction, it is possible to determine the direction and distance to which the object is moved. In one embodiment, a plurality of single-axis accelerometers 7 is utilized with a monitoring device 20. In another embodiment, at least one 2-axis and/or 3-axis accelerometer is employed in the monitoring device. Information is transmitted, sent, relayed, transmitted, or otherwise provided to the microcontroller 9, which is programmed to utilize the information from the accelerometers to calculate at least one of the position and orientation of the hand relative to the body, especially the head and face, of the individual. FIG. 3 is a schematic that shows multiple accelerometers operably connected to a microcontroller.

In a further embodiment, a monitoring device 20 includes a feedback mechanism 11 that provides a signal 12 or notification to an individual. When hand motion is detected that indicates a movement towards the face, the feedback device can send a signal or notification that alerts the individual that they are about to touch or have touched their face. The signal or notification can be audible, visual, haptic, or some combination thereof, for example, a vibration, sound, small electric shock, light, or a change in color or configuration of the monitoring device. In one embodiment, the feedback mechanism is operably connected to the microcontroller, which controls the feedback mechanism.

The wearable monitoring device can further include an interface 15 that permits the individual to interact with and/or program the microcontroller. The interface can be utilized to program the limits or constraints for hand motion and the type, timing, and intensity of the signal 12 or notification provided when one or more hands exceed those constraints or limitations. The interface can be operably connected to the microcontroller 9, as shown, for example, in FIG. 3. In one embodiment, the interface is a device used for providing instructions to the microcontroller, including input devices, such as, but not limited to, a button, switch, screen, keyboard, mouse, touchpad, and other devices known in the art. The monitoring device can be programmable with the interface to be customized to distinct movement patterns of the individual towards the face region. In one embodiment, the interface can also be used to turn on/off the signal or notification provided by the monitoring device. For example, the monitoring device can be turned off for a set period of time when the individual is eating, drinking or grooming so there is no signal or notification. The device can also be programmed not to send a signal when the individual is eating or drinking. In one embodiment, only the device on the individual's dominant hand (e.g., the hand used to eat/drink) is turned off during eating/drinking or grooming.

In one embodiment, the monitoring device 20 of the subject invention includes a housing 3 with an attached band 5, such as for example a wristband or a ring band. The band can be adjustable. The band can be suitable for attaching the housing to the wrist or to one or more fingers and can include, for example, an elastic band, a hook and loop (VELCRO™) band, metal (e.g., titanium, steel, stainless steel, galvanized steel, aluminum, gold, silver, nickel, or platinum) leather, vinyl, rubber, nylon, wood, silicone, polyurethane, ceramic, carbon fiber, plastic or other related material that is affixed with, for example, a snap, buckle, or clasp. The types of bands can include ZULU, NATO strap, rally, oyster, president, jubilee, engineer, aviator/pilot, bund, shark mesh, Milanese, tropic, perlon, waffle, double ridge, NASA, or any derivative thereof. The band can be permanently or removably connected to the housing. The band can be an integral part of the housing or distinct from the housing.

The housing can comprise one or more materials including, but not limited to, glass, metal or metal alloys (e.g., titanium, steel, stainless steel, galvanized steel, aluminum, gold, silver, nickel, or platinum) leather, vinyl, rubber, nylon, wood, silicone, polyurethane, ceramic, carbon fiber, plastic or other related material. The housing can be formed by one or more components operably connected together, such as a front piece and a back piece or a top and bottom clamshell. Alternatively, the housing can be formed of a single piece (e.g., uniform body or unibody).

One or more accelerometers 7 can be contained within the housing 3 to provide at least one signal 12 to the microcontroller 9 indicative of a hand location. More particularly, the microcontroller can determine the position of a hand or hands relative to the head and face of the individual by utilizing the information received from the one or more accelerometers. The one or more accelerometers can be a single-axis accelerometer, 2-axis accelerometer, or a 3-axis accelerometer.

FIG. 4 illustrates an example of circuitry for a monitoring device wherein multiple accelerometers provide information to a microcontroller that subsequently processes the information to determine whether to provide a signal to the individual.

Single- and multi-axis accelerometers can detect both the magnitude and the direction of mass acceleration, as a vector quantity, and can be used to sense orientation using the direction of mass changes, coordinate acceleration, vibration, shock, and falling in a resistive medium. Micro-electro-mechanical systems (MEMS) accelerometers are small, usually between about 0.02 and about 1.0 mm in diameter.

In the simplest iteration, an accelerometer 7 behaves as a damped mass on a spring. When the accelerometer experiences acceleration, the mass is displaced to the point that the spring accelerates the mass at the same rate as the casing. The displacement is then measured to give the acceleration. An accelerometer can be a simple circuit integrated into a larger electronic device. They can comprise many different components that detect and measure displacement by creating and detecting different effects caused by the displacement, two of which are the piezoelectric effect and the capacitance sensor.

The piezoelectric effect is the most common form of accelerometer and uses microscopic crystal structures that become stressed due to accelerative forces. These crystals create a voltage from the stress, and the accelerometer senses and transmits the voltage reading to the microcontroller, which is programmed to interpret the voltage to determine velocity and orientation. The capacitance accelerometer senses changes in capacitance between microstructures located next to the device. If an accelerative force moves one of these structures, the capacitance will change, and the accelerometer senses or detects the change and transmits capacitance information to the microcontroller, which is programmed to translate that capacitance into velocity and orientation.

Under the influence of external accelerations, the proof mass deflects from its neutral position. This deflection is measured in an analog or digital manner. Most commonly, the capacitance between a set of fixed beams and a set of beams attached to the proof mass is measured. This method is simple, reliable, and inexpensive. Integrating piezo resistors in the springs to detect spring deformation, and thus deflection, is a good alternative, although a few more process steps are needed during the fabrication sequence.

Modern accelerometers are often small micro-electro-mechanical systems (MEMS) comprising a cantilever beam with a proof mass that is also known as seismic mass. Damping results from the residual gas sealed in the device.

Another, relatively new type of MEMS-based accelerometer is a thermal (or convective) accelerometer that contains a small heater at the bottom of a very small dome, which heats the fluid (gas or liquid) inside the dome, producing a thermal bubble that acts as the proof mass. An accompanying temperature sensor (like thermistor; or thermopile) in the dome is used to determine the temperature profile inside the dome, hence, facilitating the determination of the location of the heated bubble within the dome. Due to any applied acceleration, there occurs a physical displacement of the thermal bubble and it gets deflected off its center position within the dome. Measuring this displacement, the acceleration applied to the sensor can be measured. Due to the absence of a solid proof mass, thermal accelerometers yield high shock survival rating.

Most micromechanical accelerometers operate in-plane, that is, they are designed to be sensitive only to a direction in the plane of the die. Integrating two devices perpendicularly on a single die provides a two-axis accelerometer. By adding another out-of-plane device, three axes can be measured.

As shown in FIG. 3, the signal 12 of an accelerometer 7 can be transmitted to the microcontroller 9 that can also be located within the housing 3 of the monitoring device. The use of an accelerometer with the microcontroller allows the monitoring device to determine if the wearer has moved a hand to the face or head area, irrespective of the type of movement used. The combination of the accelerometer and processor can detect the movement towards the face and various types of contact on the face. These types of contact include scratching, patting, holding, caressing, rubbing, or various grooming movement, such as pinching or plucking. The movement can be performed by the hand, fingers, finger nails, wrist, forearm, or a combination thereof.

The monitoring device also includes a feedback mechanism 11 that can be entirely or partially contained within the housing. The feedback mechanism is controlled by the processor. The feedback mechanism provides an indication that the wearer is touching or is imminently going to touch his/her head based on a signal 12 received from one or more accelerometers and processed though the microcontroller.

The feedback device 11 can include, for example, a buzzer, vibrator, speaker, electric shock device, or visual alert device (e.g., LED light). In one embodiment, the intensity of the signal can be increased as the hand moves closer to the face. For example, when the hand is 6 inches away from the head, an 80 dB sound is emitted, but when the hand is close enough to touch or contact the face, a 100 dB sound can be emitted. The intensity of the notification can be increased or decreased as the hand moves towards and away from the face.

The same change in signal 12 intensity can be applied to the other types of feedback mechanism 11. For example, an electric shock can be intensified, a visual alert can get brighter, or a vibrator can produce a more intense vibration. Conversely, as the hand moves away from the head, the intensity of the signal decreases until a certain distance is achieved, such as, for example, at 3, 4, 5, 6, 7, 8, 9, 10, 11, or 12 inches away, the notification is stopped. The intensity of the notification can be decreased gradually or decreased in a step-wise fashion.

The signal 12 intensity, duration of activation, pattern, color, brightness, sensitivity, or direction can be changed. For example, the duration and intensity of the electric shock can be increased or the number of light flashes or the brightness of the LED light can be decreased. These various parameters of the notification, including the intensity, duration, pattern, color, brightness, direction and sensitivity, can be changed by the wearer using the display screen.

In certain embodiments, a monitoring device 20 of the subject invention can further include a display screen interface that permits the individual to view and modify operational information and can also provide a visual alert. The display screen can be a liquid crystal display (LCD), a light emitting diode (LED) display, organic light-emitting display, organic electroluminescence, electronic ink, flexible display, or another type of display technology or combination of display technology types.

Additionally, the wearer can interact with the screen with at least 1, 2, 3, 4, or more buttons. The interaction can be a combination of (a) button(s) and a touch screen, or the entire interaction can be performed by touchscreen. The touchscreen can be configured to receive touch input, force input, rotation input and the other related inputs from the wearer. The display screen can provide the wearer with a variety of information including the number of face touches in a designated time interval (e.g., per minute, per hour, per day, per week, per month, per year), the settings of the notification (e.g. intensity, duration, pattern, sensitivity, brightness, color, or type), battery charge status, biometrics, or other health-related tasks.

In certain embodiments, the monitoring device also includes at least one LED light at least partially contained within the housing and controlled by the controller and/or processor. The LED can be used to indicate charge status (e.g., fully charged, currently charging or partially charged), the mode of operation (e.g., on or off), or provide a notification. The LED can be a single color, or a variety of colors including white, yellow, red, green, blue, orange, or purple.

In certain embodiments, the microcontroller 9 is in operable communication with memory storage 10. The microcontroller can be configured to access the memory storage 10 having instructions stored thereon. The instructions can direct the microcontroller to perform, coordinate, or monitor one or more of the operations or functions of the monitoring device. The instructions can also direct the microcontroller control or coordinate the operations of the display screen, a force or touch input/output component, one or more accelerometers, a speaker/microphone, a biometric sensor, one or more haptic feedback devices, a buzzer, a vibrator, an odor emitting device, a shock emitting, and/or the LED light. FIG. 4 illustrates an example of the circuitry for a monitoring device 20 that includes multiple accelerometers 7, a microcontroller 9 operably communicated with memory storage 10, and a feedback mechanism 11. Also shown in FIG. 4 is an amplifier 14 that can enhance the information sent to the microcontroller from the accelerometers.

In certain embodiments, a monitoring device 20 includes a battery located within the housing. The battery can provide power to the components of the monitoring device, including the one or more accelerometers, notification device, display screen, feedback mechanism, and/or microcontroller. The battery can be removable or permanent. The battery can be rechargeable and configured to provide power while the monitoring device is being worn. The monitoring device can also be configured to recharge the battery using a wireless charging system. The type of battery can be lead acid, alkaline, NiCad, Ni-MH, Li-Ion, LiPoly, or any other related type of battery or battery chemical makeup.

In certain embodiments, the monitoring device utilizes the feedback mechanism to remind the wearer not to touch the face. The feedback mechanism can further notify the wearer of progress towards a goal or reducing touching or contact with the face. For example, the goal can be a limit to the number of times the face is touched or contacted during in a set time period. When the goal is reached, a signal can be provided to indicate the goal has been reached. The signal indicating that the goal has been reached can be the different from a signal indicating that the hand is in proximity to the head or face.

In certain embodiments, a monitoring device 20 is incorporated into a watch, bracelet, wristband, ring band, ring cuff, or other object that can be attached to or worn on or in proximity to a hand. Ideally, the other object can accommodate a microcontroller 9, one or more accelerometers, and a feedback mechanism 11. It can also be beneficial if the other object can incorporate an interface 15 for accessing the microcontroller.

In one embodiment, the microcontroller 9 is programmed with the interface 15, such as a display screen, to customize the monitoring device to recognize distinct movement patterns of the hand or wrist through threshold memorization based on the signals provided by the accelerometer. In other embodiments, the monitoring device is provided with preprogrammed settings to recognize common movement patterns of touching the face.

Once the programmed movement pattern is detected the monitoring device can provide a signal to the wearer. If the movement persists, the signal can change in intensity, such as, for example, increasing the decibels, brightness, electrical current (amperes), or force of vibration. If the movement stops and/or reverses (e.g., moving the hand away from the head or face), the signal can be stopped, the intensity of the notification can be decreased, or the intensity can be decreased until the monitoring device determines the hand is not in proximity to the face or head.

In certain embodiments, the monitoring device 20 can be limited to the number of signals provided in a set amount of time. For example, the microcontroller can be programmed to provide not more than 1 notification per second, not more than one signal per 15 seconds, not more than one signal per 30 seconds, not more than one signal per minute, or not more than one signal per a greater time interval.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 can comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1: Wrist-Worn Monitoring Device

A monitoring device includes a housing with an adjustable wristband attached thereto. The monitoring device is placed on the wrist and secured with the adjustable wristband, preferably as close to the hand as possible. An accelerometer is disposed within the housing to provide a signal indicative of the motion of the hand in space relative to the body of an individual. The accelerometer uses gravity as an input vector to determine the motion of the hand in the space around the body of the individual. The accelerometer can be a two-axis accelerometer or a three-axis accelerometer, which can be obtained, for example, from Analog Devices, Inc. (Norwood, Mass.) and Bosch Sensortec GmbH (Germany).

A microcontroller disposed within the housing is operatively coupled to the accelerometer. The accelerometer sends information to the microcontroller pertaining to the motions of the monitoring device, which corresponds to the location of the hand. The individual initially programs the microcontroller, by depressing a button to initiate a programming procedure. When the programming procedure is initiated, the microcontroller will start to recognize and store information about the normal motions the individual, when not touching the head or face. Once the individual has performed sufficient motions the button is pressed again to stop the programming procedure. The microcontroller will then be programmed with one or more boundaries that define the space around the individual in which the monitoring device, and consequently the hand, is moved during normal activity. Since the head and face were not touched during the programming process, that area will not be considered within the boundaries of normal motion.

The monitoring device can further include at least one dual-color LED viewable by the individual and operatively coupled to the microcontroller. When the monitoring device is in operation and within the programmed boundaries, the microcontroller causes the LED to show a first color (green). When the microcontroller determines that the monitoring device is approaching the programmed boundaries, such as moving towards the head or face, the microcontroller cause the LED to present a second color (red). The color of the LED can act as a warning to the individual that the hand is approaching the head or face and deter completion of the movement.

What is claimed:

1. A monitoring device, configured to be worn on or in proximity to a hand, comprising:
at least one accelerometer that senses motion of the hand;
a microcontroller, in operable communication with the accelerometer, programmable with one or more boundaries and that receives information from the accelerometer pertaining to the motion of the hand;
a transmitter, in operable communication with the microcontroller, for transmitting a position information signal pertaining to the location of the monitoring device; and
a feedback mechanism controlled by the microcontroller that provides a signal when the microcontroller determines that the hand approaches or passes a programmed boundary.

2. The monitoring device according to claim 1, wherein the monitoring device is configured to be worn on a wrist or on one or more fingers.

3. The monitoring device according to claim 1, further comprising an interface for programming the microcontroller with the programmed boundaries.

4. The monitoring device according to claim 1, further comprising a receiver for receiving a position information signal from a second monitoring device, configured to be worn on or in proximity to a second hand.

5. The monitoring device according to claim 1, wherein the feedback mechanism is at least one of visual, audible, and haptic.

6. A method for inhibiting hand contact with the face of a subject, the method comprising:
affixing on or in proximity to a hand of the subject a monitoring device comprising,
at least one accelerometer that detects motion of the hand;
a microcontroller, in operable communication with the accelerometer, programed with one or more boundaries and that receives information from the accelerometer pertaining to the motion of the hand;
a transmitter, in operable communication with the microcontroller, for transmitting a position information signal pertaining to the location of the monitoring device; and
a feedback mechanism controlled by the microcontroller that provides a signal when the microcontroller determines that motion of the hand approaches or passes a programmed boundary;
and wherein the subject moves the hand away from the programmed boundary in response to the signal.

7. The method according to claim 6, wherein the monitoring device further comprises an interface and the method further comprises utilizing the interface to program the microcontroller.

8. The method according to claim 6, further comprising:
a) affixing a second monitoring device on or in proximity to a second hand of the subject, wherein the second monitoring device comprises at least one accelerometer that senses motion of the hand;
a microcontroller, in operable communication with the accelerometer, programmable with one or more boundaries and that receives information from the accelerometer pertaining to the motion of the hand;
a transmitter, in operable communication with the microcontroller, for transmitting a position information signal pertaining to the location of the monitoring device; and
a feedback mechanism controlled by the microcontroller that provides a signal when the microcontroller determines that the hand approaches or passes a programmed boundary; and
b) programming the microcontroller of the second monitoring device with one or more boundaries; and wherein the subject moves the second hand away from the programmed boundary in response to the signal.

9. The method according to claim 8, wherein the microcontroller in the monitoring device is further programmed to compare the information from the one or more accelerometers with the position information signal received from the second monitoring device and determine when the hands are arranged in the one or more pre-defined orientations, to prevent a signal from being provided by the feedback mechanism.

10. The method according to claim 6, wherein the feedback mechanism provides a signal that is at least one of visual, audible, and haptic.

11. The method according to claim 6, wherein the one or more programmed boundary excludes a space around the face, such that motion of the hand towards the face causes the feedback mechanism to send a signal.

12. The method according to claim 6, wherein the device is turned off during eating and/or drinking.

13. The method according to claim 6, wherein the device is programmed not to send a signal when the hand approaches the mouth.

14. The method according to claim 12, wherein only a device on the subject's dominant hand is turned off during eating and/or drinking.
